(19) United States
(12) Patent Application Publication
(10) Pub. No.: US 2019/0225993 A1
(43) Pub. Date: Jul. 25, 2019

(54) SYSTEMS AND METHODS FOR MICROBIAL PRODUCTION

(71) Applicant: ICELL SUSTAINABLE (SHANGHAI) NUTRITION CO., LTD, Shanghai (CN)

(72) Inventors: Weiwei ZHAO, Shanghai (CN); Jianhua SONG, Shanghai (CN); Xuefeng WANG, Shanghai (CN); Qingye JIANG, Shanghai (CN)

(21) Appl. No.: 16/253,240

(22) Filed: Jan. 22, 2019

(30) Foreign Application Priority Data
Jan. 22, 2018 (CN) 201810057792.4

(51) Int. Cl. C12P 1/04

(52) U.S. Cl. CPC C12P 1/04; C05B 17/00; C12N 2500/34

(57) ABSTRACT

Methods to produce microbial biomass are provided. The methods involve the inoculation of a liquid growth medium including a controlled substrate with a microbial inoculum and incubation under conditions that are suitable for the production of biomass. The biomass and/or the liquid growth medium may be used to produce valuable products including food and feed products.

This application claims priority to Chinese Patent Application Ser. No. CN201810057792.4 filed on 22 Jan. 2018.

### BACKGROUND

The following paragraphs are intended to introduce the reader to the more detailed description that follows and not to define or limit the claimed subject matter of the present disclosure.

The manufacture of industrially made products commonly yields polluting organic by-products. Thus, for example, benzene and benzene derivatives are by products generated in the processing of petroleum products, coking of coal and the production of xylene and toluene and other aromatic products, and are known to be carcinogens (IPCS(1993) Benzene: Geneva World Health Organization, International Programme on Chemical Safety; Environmental Health Criteria 150). In order to limit the pollution challenges associated with organic by-products, waste treatment facilities are designed to process these organic products and yield alternate products that may be safely disposed. More recently, waste treatment facilities have started to focus on conversion of organic by-products into products that have commercial value, thus avoiding disposal and the associated disposal costs altogether (see e.g. U.S. Pat. No. 7,931,806). One approach to processing organic by-products harnesses the ability of microbial organisms to metabolize complex organic molecules and convert these molecules into simple molecules, such as carbon dioxide and water. Nevertheless, some substrates remain recalcitrant to microbial conversion into products that are safe to dispose, or into products that increase the commercial value of the manufacturing process. Thus, for example, condensed syrup, a by-product in the manufacture of corn ethanol, is deemed unsuitable as a substrate for microbial production for at least the following reasons. Firstly, the biological oxygen demand of the material typically exceeds 500,000 mg/L, thereby rendering the substrate unsuitable for aerobic microbiological proliferation. Secondly, condensed syrup has a very high viscosity, often within the range of 3,000-4,000 centipoise. This makes handling of the material difficult, and renders the substrate unsuitable as an aerobic growth medium due to the fact that mixing, aerating, and pumping of the material is difficult and would lead to a non-homogeneous growth environment. Thirdly, condensed syrup is the by-product of yeast fermentation of corn sugars. It is therefore a mixture of compounds that are more recalcitrant to microbial fermentation.

Other polluting organic byproducts are also known, with characteristics similar to those of condensed syrup. For example, palm oil mills produce an effluent stream which is similarly very high in biological oxygen demand (typically 30,000-40,000 mg/L), cane- and beet sugar manufacturers produce vinasses with biological oxygen demands normally exceeding 25,000 mg/L, and biodiesel manufacturers produce significant quantities of low-quality glycerin, again with a very high biological oxygen demand exceeding 500,000 mg/L. Such organic waste streams share the characteristic of being unsuitable for aerobic microbiological proliferation, and cannot be conveniently disposed of, e.g. In a wastewater stream, without disastrous environmental consequences. Accordingly, such organic wastes pose difficult disposal and environmental problems as they cannot be handled in conventional waste streams.

Existing waste product treatment facilities generally receive industrial waste products for processing comprising a wide range of constituents in a wide range of concentrations. The extreme diversity of waste products makes the use of these waste products as a substrate for microbial growth, and the design and operation of microbial processes for the treatment of industrial waste, challenging.

Further, existing waste treatment processes generally involve the collection of waste products, e.g. waste water, that are substantially contaminated, for example, with microbial organisms and/or toxins, making these waste products unsuitable for the preparation, for example, of human food products.

Therefore, there is an unmet need in the art to develop further processes and substrates that may be metabolized by microbial organisms.

### SUMMARY

The following paragraphs are intended to introduce the reader to the more detailed description that follows and not to define or limit the claimed subject matter of the present disclosure.

In one aspect, the present disclosure relates to the production of microbial biomass. In another aspect, the present disclosure relates to the production of microbial biomass in a medium including controlled substrates.

Accordingly, the present disclosure provides, in at least one embodiment, a method comprising:

receiving a growth medium including a controlled substrate; treating the growth medium to generate a treated growth medium, the treated growth medium having a biological oxygen demand (BOD) value from about 10,000 mg BOD/L to about 90,000 mg BOD/L;

inoculating the treated growth medium with a microbial composition to generate an inoculated medium, the microbial composition including two or more microbial species, each microbial species independently selected from the group of microbial families consisting of Sphingobacterisceae, Comamonadaceae, Xanthomonadaceae, Microbacteriaceae, Flavobacteriaceae, Alcaligenaceae, Porphyromonadaceae, and Saprospiraceae;

incubating the inoculated medium under conditions suitable for microbial growth to generate an incubation product; and extracting biomass from the incubation product.

In some embodiments, the controlled substrate is condensed distillers syrup. In some embodiments, the controlled substrate is palm oil mill effluent. In some embodiments, the controlled substrate is vinasse. In some embodiments, the controlled substrate is glycerin.

The present disclosure further provides, in at least one embodiment a method comprising receiving a growth medium including a controlled substrate; treating the growth medium to generate a treated growth medium, the treated growth medium having a biological oxygen demand (BOD) ranging from about 10,000 mg BOD/L to about 1,000,000 mg BOD/L; inoculating the treated growth medium with a microbial composition to generate an inoculated medium; incubating the inoculated medium under conditions suitable for microbial growth to generate an incubation product, including maintaining the pH of the inoculated medium between about 6.5 and about 7.5; and extracting biomass from the incubation product.

In some embodiments, the conditions suitable for microbial growth further include maintaining the dissolved oxygen level at a concentration greater than approximately 3.0 mg/L. In some embodiments, the conditions suitable for microbial growth further include maintaining an MCRT of 3 days or less. In some embodiments, the microbial composition comprises an extremophilic microbial species. In some embodiments, the microbial composition comprises an thermophilic microbial species.

The produced biomass as well as the liquid medium may be used to prepare valuable products, including food and feed products. The present disclosure further provides, in at least one embodiment, a method for producing phosphorus comprising: (a) providing a growth medium including a controlled substrate; (b) providing a microbial inoculum; (c) inoculating the growth medium with the microbial inoculum; (d) growing microbial cells under conditions that permit the conversion of the controlled substrate and producing bacterial mass and depleted growth medium; (e) separating the microbial mass from the depleted growth medium; and extracting phosphorus from the depleted growth medium;

Other features and advantage or the present disclosure will become apparent from the following detailed description. It should be understood, however, that the detailed description, while indicating preferred implementations of the present disclosure, are given by way of illustration only, since various changes and modification within the spirit and scope of the disclosure will become apparent to those of skill in the art from the detailed description.

### DETAILED DESCRIPTION OF THE INVENTION

Aspects disclosed herein provide systems, methods, and compositions for converting waste streams containing controlled substrates, which otherwise cannot be handled by conventional waste management processes due to their high biological oxygen demand and other physical characteristics (e.g. viscosity), to a high-value source of protein, nucleic acids, nucleotides, vitamins and other nutritional components for animal and/or human use.

As hereinbefore mentioned, various large-scale processes such as ethanol fermentation, palm oil milling, sugar products, and biodiesel production produce high biological oxygen demand by products such as condensed syrup, palm oil mill effluent, vinasses, and glycerin that are unsuitable for supporting aerobic microbial growth. Such substrates cannot be practically or legally disposed of in wastewater streams in the US because reducing the biological oxygen demand would require dilution with impractical volumes of clean water, and because of the high biological oxygen demand, it would not be acceptable to add such materials to the wastewater stream directly. In addition, many of these substrates have other characteristics such as high viscosity, low water activity, etc. That make handling difficult and inhibit microbiological growth, particularly aerobic organisms, due to difficultly in supplying sufficient oxygen. Anaerobic processes are a viable option for reducing the biological oxygen demand (BOD) of the water substrates but anaerobic metabolism is not optimal for the production of a substantial quantity of bacterial mass containing protein, nucleic acids, nucleotides, vitamins and other nutritional components due to the low yield of the process (generally on the order of 0.2 pounds of cellular production per pound of BOD input and is in contrast to at least 0.5 pounds of cellular production per pound of BOD input in anaerobic system), and the fact that these system are normally “fixed film”. This latter point means that cells are growth on fixed media within the anaerobic contact vessel and are of unknown age. The average protein and nutritional value of the cells produced is therefore low and the ash content is high because the average age of the cells relatively old. In anaerobic lagoons, the low yield coupled with the inability to control cellular age, results in low protein concentration in the cellular product.

As hereinbefore mentioned, various large-scale processes such as the biological conversion of growth media containing a high concentration of BOD also results in the production of heat energy as the BOD is converted into cellular biomass. As a result, biological conversion systems that utilize mesophilic bacteria necessarily, include a means for heat dissipation to ensure that the process stays within the ideal temperature range of approximately 20° C. to 45° C. An example of an appropriate heat dissipation systems is a heat exchanger and cooling tower. An appropriately sized heat dissipation systems will effectively reduce heat accumulation and will allow the culturing of mesophilic microorganisms. The costs associated with the construction and maintenance of an effective heat dissipation system render the mesophilic process more costly versus one that can operate at temperatures above the mesophilic range. An alternative embodiment therefore includes the utilization of thermophilic microorganisms that function optimally within a temperature range. The use of thermophilic microorganisms therefore results in the avoidance of costs related to heat dissipation thereby resulting in a more economic production process.

Such organic byproducts present difficult waste disposal problems because they are not readily adaptable to conventional waste treatment process, and /or would cause substantial environmental problems, if treated using conventional methods. In various embodiments, the present invention provides methods for converting such byproducts into high-value-added animal and/or human grade protein sources. As described herein, organic byproducts such as condensed syrup produced as a byproduct from ethanol fermentation, palm oil effluent produced as a byproduct of palm oil production, and glycerin, produced as a byproduct of biodiesel production, and other similar organic byproducts, typically characterized by high biological oxygen demand, and/or high viscosity, and/or low water activity, and other characteristics (such as low pH) which render ready disposal difficult, can be converted to controlled substrates for the production of microbiological protein suitable for animal or human use.

Methods of producing food-grade microbial protein from wastewater streams has been described, for example in U.S. Pat. No. 7,931,806, herein incorporated by reference in its entirety. In contrast to the controlled substrates of the process(es) described herein , wastewater streams from food processing plants typically have relatively low biological oxygen demand of less than about 500 mg BOD/L, and it is relatively easy to use such wastewater streams directly to support the growth of aerobic microorganisms. However, the composition of wastewater can vary significantly over time, and can therefore be difficult to control the growth of microorganisms to provide high-quality, food grade protein. In various embodiments of the present invention, specific organic waste streams, which typically cannot be disposed of in a wastewater stream (as described herein) are utilized as growth substrates for microorganisms. These organic waste streams are characterized by extremely high biological oxygen demand, often high viscosity, low pH, low water activity, or other physical and chemical characteristics which render them otherwise unsuitable to support the growth of aerobic microorganisms.

For example, some of these substrates, such as condensed syrup, glycerin, etc. are effectively shelf-stable and resist or inhibit microbial growth. As such, these substrates are not conventionally considered suitable for use as growth media for microorganisms. In the case of byproducts such as glycerin, most generators try to create value-added products with it, i.e, add value by further refining it, etc. because such refining is necessary in order to create a market for the product. As described herein, in various embodiments, such byproducts can be modified (e.g., by dilution, pH adjustment, adjustment with micronutrients, etc.) to provide a suitable substrate for microbial growth, which can be used to provide food grade protein. Such substrates differ from growth media that are used, e.g. in biotechnology to support the growth of cell cultures and other microorganisms. Conventional growth media utilize relatively expensive nutrients that are recognized for their suitability to support bacterial growth.

The present disclosure provides, in at least one embodiment, a liquid medium comprising a controlled substrate as a growth substrate/medium for microbial production. In some embodiments, methods for processing industrial waste products and preparing microbial mass are provided. The methods and compositions herein provided involve the processing and use of industrial waste products in a manner heretofore not known. The methods and compositions of the present disclosure are beneficial in that they involve the use of a consistently constituted industrial waste product as a substrate for microbial production, thereby substantially facilitating the design and operation of waste treatment operations. Notably, the methods of the present disclosure minimize the need to use a clarifier and improve the recycling of water used in the waste treatment facility and reduce the amount of waste water generated by the waste treatment facility. Additionally, this disclosure includes an explanation of the utility of bacteria that are applicable to the process including those capable of metabolizing cellulose and starch and those that are able to function optimally at temperatures suitable for thermophilic bacteria. Furthermore, the methods and compositions of the present disclosure permit the use of industrial waste products in the manufacture of human food products, as well as animal feed products. Furthermore, the compositions and methods herein provided permit the use of industrial waste products heretofore deemed unsuitable as substrates for microbial production.

Various compositions and methods will be described below to provide examples of an embodiment of each claimed subject matter. No embodiment described below limits any claimed subject matter and any claimed subject matter may cover methods, processes, compositions or systems that differ from those described below. The claimed subject matter is not limited to compositions or methods having all of the features of any one composition, method, system or process described below or to features common to multiple or all of the compositions, systems or methods described below. It is possible that a composition, system, method or process described below is not an embodiment of any claimed subject matter. Any subject matter disclosed in a composition, system, method or process described below that is not claimed in this document may be the subject matter of another protective instrument, for example, a continuing patent application, and the applicants, inventors or owners do not intend to abandon, disclaim or dedicate to the public any such subject matter by its disclosure in this document.

It should be noted that terms of degree such as “substantially”, “essentially” “about” and “approximately” as used herein mean a reasonable amount of deviation of the modified term such that the end result is not significantly changed. These terms of degree should be construed as including a deviation of the modified term if this deviation would not negate the meaning of the term it modifies.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

As used in this specification and the appended claims, the singular forms “a”, “an”, and “the” include plural referents unless the content clearly dictates otherwise Thus, for example, reference to “a microbial cell” includes two or more such cells, reference to “a substrate” includes reference to mixtures of two or more substrates, reference to “a product” includes two or more such products, and the like.

All publications, patents and patent applications are herein incorporated by reference in their entirety. Unless defined otherwise, all technical and scientific terms used herein shall have the same meaning as commonly understood by one of ordinary skill in the art to which the disclosure pertains. The following terms shall be understood to have the following meanings.

The term “microbial inoculum” as used herein, refers to a quantity of live microbial cells used to initiate microbial growth in a growth medium. The inoculum may comprise one or more microbial species.

The term “growth medium” as used herein refers to a liquid medium comprising a mixture of chemical compounds, that together function as nutrients permitting growth of microbial cells.

The term “food, beverage or biofuel processor by-product” as used herein refers to a by-product or waste product obtainable or obtained from a food or biofuel production process. Examples of food, beverage or biofuel processor by-products are condensed distillers syrup, glycerin, vinasse and palm oil mill effluent.

The term “food grade conditions” as used herein is a qualitative term referring to storing and/or operating conditions designed such that products (e.g. food, beverage or biofuel processor by-products) remain substantially free from contaminating substances, such as toxic compounds and harmful microbial organisms, thereby permitting use of such products in processes for the manufacture of food or feed products.

The terms “Biological Oxygen Demand” or “BOD” as interchangeably used herein refer to the quantity of oxygen required to microbially degrade organic carbon.

The term “controlled substrate” as used herein refers to a consistently constituted process by-product which has been treated and maintained in such a manner that it becomes suitable as a growth substrate for a microbial production process. The term “controlled substrate” as used herein additionally refers to a substance that is unsuitable for disposal into typical wastewater streams due to high biological oxygen demand (BOD). The BOD of the controlled substrate can be in a range from about 100,000 mg BOD/L to about 1,000,000 mg BOD/L, including all values and subranges in between. The viscosity of a controlled substrate can be in the range from about 1 centipoise to 4,000 centipoise. In examples where the controlled substrate is condensed distillers syrup, glycerin or vinass, the viscosity can be from about 3,000 centipoise to 4,000 centipoise.

The terms “mesophilic”, “mesophile” or “mesophiles” refer to the group of bacteria that are known to inhabit environments that are normally between about 20° C. and about 45° C.

The terms “thermophilic”, “thermophile” or “thermophiles” refer to the group of bacteria that are known to inhabit environments that are normally between about 41° C. and about 100° C. or more.

As hereinbefore mentioned, the present disclosure provides, in at least one embodiment, a liquid medium comprising a controlled substrate as a growth substrate for microbial production.

Accordingly, a quantity of controlled substrate is contacted with medium constituents to form a growth medium comprising the controlled substrate. In some embodiments, at least one of the medium constituents is a liquid constituent, such as water. The medium constituents may be any medium constituents suitable for microbial growth. The medium constituents, in addition to a liquid constituent, can also include salts, trace amounts of metal elements, proteins, lipids and the like, and can further include exogenous dissolved organic carbon in a form that can be utilized by bacterial cells such as carbohydrates including sugars, starches, and similar compounds, wherein such dissolved organic carbon is provided by the controlled substrate. The medium constituents, following contacting with the controlled substrate, can include significant amounts of organic carbon (e.g. more than 10,000 mg/L as measured by BOD and often more than 500,000 mg/L as measured by BOD), as the controlled substrate can serve as an organic carbon substrate to the medium. The controlled substrate may be provided in any form, e.g. as a liquid, or a solid, for example, a dry powder, and is typically contacted with the medium constituents under conditions that allow for a more or less homogenous dispersal of the controlled substrate into the liquid medium. Such conditions include a viscosity that allows substantially complete mixing and diffusion of oxygen and/or ambient air (less than approximately 30 centipoise at 25° C. or higher temperatures) When provided as a dry ingredient, the controlled substrate may initially be mixed with other dry ingredients.

In some embodiments, when provided as a liquid, the controlled substrate may be mixed into the liquid medium, or, in other embodiments, the controlled substrate may be provided in a sufficient quantity of liquid constituent so that it is not necessary to add further liquid constituents. Typical final concentrations of the controlled substrate in the growth medium may vary, but the BOD of the resulting growth medium, in some embodiments, is from about 15,000 mg BOD/L to 25,000 mg BOD/L.

In some embodiments, the substrates which have high BOD, for example in excess of about 100,000 mg BOD/L, and up to about 1,000,000 mg BOD/L, or more are diluted down to a target BOD of about 10,000-90,000 mg BOD/L, more particularly about 10,000-40,000 mg BOD/L. In some embodiments, the BOD of the substrate is adjusted to about 10,000, about 20,000, about 30,000, about 40,000, about 50,000, about 60,000, about 70,000, about 80,000, or about 90,000 mg BOD/L, inclusive of all ranges and subranges there between.

The controlled substrate in accordance herewith is prepared from a food, beverage or biofuel processor by-product. Accordingly the present disclosure further comprises a method of preparing a controlled substrate comprising: (a) providing a food, beverage or biofuel processor by-product; and (b) treating the food, beverage or biofuel processor by-product to obtain a controlled substrate.

The treatment of the food, beverage or biofuel processor by-products is directed at obtaining or maintaining a substrate that is chemically consistent, i.e a substrate that is comprised of approximately the same constituents in the same relative concentrations. In particular embodiments, the treatment of the food, beverage or biofuel processor by-product to obtain a controlled substrate comprises: (a) maintenance of the food, beverage or biofuel processor by-product under food-grade conditions; (b) reducing the biological oxygen demand; (c) diluting the food, beverage or biofuel processor by-product; and/or (d) processing the food, beverage or biofuel processor by-product by (i) enzymatic treatment of the food, beverage or biofuel processor by-product and/or or (ii) comminuting the food, beverage or biofuel processor by-product.

In some embodiments, the food, beverage or biofuel processor by-products include condensed syrup, glycerin, palm oil mill effluent (POME), vinasse, and/or combinations thereof.

The maintenance of the food, beverage or biofuel processor by-product or controlled substrate under food-grade conditions could include (but are not limited to)steps such as (i) segregating different by-products to maintain consistency of an individual by-product, (ii) diverting waste products to sewers, treatment systems, and/or isolated collection systems, (iii) conveying the food, beverage or biofuel processor by-product or controlled substrate with food-grade pumps, piping, and other food-grade materials, (iv) utilizing lined and covered basins or enclosed tanks that isolate the food, beverage or biofuel processor by-product or controlled substrate from the air or other uncontrolled environments, (v) inoculating the food, beverage or biofuel processor by-product medium with food-grade microorganisms and/or (vi) utilizing food-grade nutrient, chemicals, or other inputs to the food, beverage or biofuel processor by-products.

In some embodiments, dilution of the food, beverage or biofuel processor by-product may be achieved by mixing the food, beverage or biofuel processor by-product with a suitable diluent, such as fresh water or water that is produced during the microbial harvesting process. In the latter case, this recycled water may be partially or completely filtered (e.g., using reverse osmosis filtration) to remove microbial metabolites.

In some embodiments, the biological oxygen demand may be reduced by mixing the food, beverage or biofuel processor by-product with a suitable diluent, such as fresh water or water that is produced during the microbial harvesting process.

In embodiments hereof wherein the food, beverage or biofuel processor by-products are processed, such processing may include the addition of enzymes including amylase, cellulase, lipase, hemicellulase, dextranase or other enzymes that enzymatically digest the food, beverage or biofuel processor by-products in order to enhance the availability to the microbiological community that utilizes controlled substrate, or to improve material handling characteristics. Additionally, digestion or “steam explosion” technologies may be employed for this purpose. Thus, for example the use of dextranase results in the reduction of dextran levels in dextran-rich food, beverage or biofuel processor by-products, which improves the material handling characteristics of food, beverage or biofuel processor by-products and/or the controlled substrate. The food, beverage or biofuel processor by-products may also be comminuted e.g. by milling or grinding the food, beverage or biofuel by-product using for example a colloid mill, cone mill, or other type of wet mill that reduces the particle size of the food, beverage or biofuel processor by-product. In particular embodiments, comminution results in a size reduction rendering at least 50% of the particles into colloidal size, i.e. Approximately 0.45 microns or smaller. Alternatively, comminution results in a size reduction that renders at least 50% of the particles into a size small enough to be fully metabolized by the bacterial inoculum within a period of approximately 24 hours. Comminution of the food, beverage or biofuel by-product may be performed before enzyme treatment, after enzyme treatment, or in lieu of enzyme treatment.

The present disclosure further provides methods for microbial production using a controlled substrate as a growth substrate. Accordingly, the present disclosure further provides a method of microbial production comprising: (a) providing a growth medium comprising a controlled substrate; (b) providing a microbial inoculum; (c) inoculating the growth medium with the microbial inoculum; and (d) growing microbial cells under conditions that permit the conversion of controlled substrate by the microbial cells.

In accordance herewith the growth medium is contacted with a microbial inoculum. The inoculum can be provided in liquid form and injected into the growth medium (e.g., one or several milliliters of inoculum per liter of liquid medium). The inoculum comprises at least one microbial species. In particular embodiments, the inoculum comprises a microbial community comprising a plurality of microbial species, e.g., aerobic, heterotrophic and non-pathogenic microbial species, such as may, for example, be found in the return activated sludge or mixed liquor suspended solids from a wastewater treatment plant at a human-food, beverage or biofuel manufacturer. In alternative embodiments, the microbial community comprises microbial species able to tolerate growth temperatures within the range of thermophiles. In yet other embodiments, the microbial community comprises microbial species that are able to metabolize cellulose, starch, and mixtures thereof.

In one embodiment, the foregoing process is performed in a small-volume reactor. The outflow of the small volume reactor, containing the microbial cells, is subsequently introduced into to a full-scale reactor. This process may be configured for batch or continuous operation.

In order to grow the microbial cells, the cells and medium are brought and maintained under conditions that permit the microbial cells to metabolize and multiply in the medium. Such conditions generally include a variety of aerobic conditions including (a) the addition of oxygen to achieve dissolved oxygen levels in excess of 1.5 mg/L, e .g.1.5-2.0 mg/L; 2.0-2.5 mg/L, or preferably greater than 2.5 mg/L or preferably greater than3.0 mg/L, (b) the addition of nitrogen and phosphorus in a ratio to BOD of 100 mg/LBOD: 10 mg/L N:2 mg/L P, wherein nitrogen is preferably added in a reduced form such as found in ammonia compounds or urea, and wherein phosphorus is preferably added in the form of phosphate, such as that found in phosphoric acid, (c) the amendment of micronutrients including, without limitation: aluminum (e.g. at a BOD normalized dose of between about 60 mg/day /lb BOD/day and 285 mg/day/lb BOD/day); boron (e.g. at a BOD normalized dose of between about 115 mg/day/lb BOD/day and 300 mg/day/lb BOD/day), cobalt (e.g. at a BOD normalized dose of between about 50 mg/day/lb BOD/day and 500 mg/day/lb BOD/day), magnesium (e.g. at a BOD normalized dose of at least about 100 mg/day/lb BOD/day, manganese (e.g. at a BOD normalized dose of between about 65 mg/day/lb BOD/day and 220 mg/day/lb BOD/day)and zinc (e.g. at a BOD normalized dose of between about 115 mg/day/lb BOD/day and 275 mg/day/lb /day), (d) substantially complete mixing due to the mixing action of the compressed air being delivered to the growth vessels as well as paddle mixers, jet mixers, and the like, (e) the continuous inflow of nutrient-amended by-product or controlled substrate in order to maintain the biological oxygen demand and nutrient concentrations of the growth vessel at the desired level and to achieve chemostatic conditions within the growth vessel, and (f) the maintenance of a high concentration of microbiological cells within the growth vessel that is in excess of 5,000 mg/L biological solids, 10,000 mg/L, 15,000 mg/L, or most preferably in excess of 20,000 mg /L biological solids in the growth vessel.

The utilization of BOD by microbes results in the production of heat to the biological process. An approximate value of the net energy liberated by the metabolism of BOD by bacteria is 4,000 British Thermal Units (BTUs) per pound of BOD. The utilization of growth media with high concentrations of BOD will therefore result in a significant production of heat. Mesophilic bacteria, those functioning optimally in the temperature range between 20° C. and 45° C., will therefore require that the temperature of the growth medium is cooled. This cooling can be achieved using for example heat exchangers and cooling tower. Thermophilic bacteria, those functioning optimally in the temperature range between 40° C. and over 100° C., will therefore not require cooling equipment. The advantages of using thermophilic bacteria in the process therefore include cost avoidance associated with building and maintaining the cooling equipment.

In accordance herewith during the growing process the controlled substrate is consumed by the microbial cells, and breakdown products are formed in the growth medium. It is noted, however, that in certain embodiments hereof a continuous microbial production system is provided. In such continuous microbial production system the controlled substrate is continuously supplied to the production system and thus the concentration of the controlled substrate in the production system remains substantially unchanged. It is further noted that in some embodiments, e.g. in continuous microbial production systems involving the recirculation of diluent water, an increase in breakdown products in the medium necessitates the removal of such breakdown products. Such removal may be achieved by collecting the growth medium and/or diluent water, separating the microbial cells from the growth medium and disposing of the growth medium and/or diluent e.g. by discharge in a waterway or underground injection. In other embodiments methodologies such as reverse osmosis or ultrafiltration may be used to remove breakdown products.

In accordance with a further embodiment of the present disclosure , the microbial mass may be harvested. Accordingly, the present disclosure further provides a method for producing a microbial mass comprising: (a) providing a growth medium including a controlled substrate; (b) providing a microbial inoculum; (c) inoculating the growth medium with the microbial inoculum; (d) growing microbial cells under conditions that permit the conversion of the controlled and producing bacterial mass; and (e) harvesting the microbial mass.

The present disclosure further provides a method for producing a microbial mass comprising: (a) providing a growth medium comprising a controlled substrate; (b) providing a mesophilic microbial inoculum; (c) inoculating the growth medium with the mesophilic microbial inoculum; (d) dissipating heat from the system; (e) growing microbial cells under conditions that permit the conversion of the controlled and producing bacterial mass; and (f) harvesting the microbial mass.

The present disclosure further provides a method for producing a microbial mass comprising: (a) providing a growth medium comprising a controlled substrate; (b) providing a thermophilic microbial inoculum; (c) inoculating the growth medium with the thermophilic microbial inoculum; (d) growing microbial cells under conditions that permit the conversion of the controlled at temperatures exceeding 40° C. and producing bacterial mass; and (e) harvesting the microbial mass.

Harvesting is generally performed following depletion of the growth medium of one or more nutrients. Microbial harvesting involves separation of the microbial mass from the growth medium. This may for example be accomplished by centrifugation, or other solid liquid separation methodology, resulting in sedimentation of the microbial cells. Once separated the liquid fraction and solid fraction may be separately treated, as liquid for recirculation or disposal and solid biomass, e.g protein aceous biomass respectively.

In other embodiments of the present disclosure, the microbial mass and/or the liquid medium may be used to extract valuable products, such as proteins, lipids, nucleic acids, vitamins and other compounds. Such valuable products further include polysaccharides, including without limitation mannans and beta glucans, polymeric compounds, including without limitation, plastic polymers, including, without limitation polyhydroxyalkanoate (PHA) polyhydroxybutyrate (PHB) containing plastic polymers, nucleotides, coenzymes, and choline. Upon extraction these products may be formulated for end use, for example as a feed product, or a fertilizer product, or a biodegradable plastic.

In other embodiments, the microbial mass and/or the liquid medium may be used to extract minerals. In one embodiment, a mineral that is extracted is phosphorus In another embodiment, the controlled substrate is condensed distillers syrup and the microbial mass and/or liquid medium is used to extract phosphorus. The extracted phosphorus may be used, for example, in the formulation of a fertilizer product Accordingly, the present disclosure further provides a method for producing phosphorus comprising: (a) providing a growth medium including a controlled substrate; (b) providing a microbial inoculum; (c) inoculating the growth medium with the microbial inoculum; (d) growing microbial cells under conditions that permit the conversion of the controlled substrate and producing bacterial mass and depleted growth medium; (e) separating the microbial mass from the depleted growth medium; and (f) extracting phosphorus from the depleted growth medium.

In some embodiments, the controlled substrate is a condensed distillers syrup. In some embodiments, the controlled substrate is a substrate rich in phosphorus. In some embodiments the controlled substrate is a substrate containing at least about 0.4%,0.5%, 0.6%, 0.7%, 0.8%, 0.9%, 1.0%, 1.1%, 1.2%, 1.3%, 1.4%, 1.5%, 1.6%, 1.7%, 1.8%, 1.9% or at least about 2% (w/w) phosphorus. In some embodiments, the controlled substrate is a condensed distillers syrup containing at least about 0.4%,0.5%, 0.6%, 0.7%, 0.8%, 0.9%, 1.0%, 1.1%, 1.2%, 1.3%, 1.4%, 1.5%, 1.6%, 1.7%, 1.8%, 1.9% or at least about 2% (w/w) phosphorus.

Growth medium from which phosphorus is removed may be readily disposed in view of its low phosphorus content e.g. by discharge in a waterway or underground injection. It is noted that the removal of phosphorus from aqueous waste products is desirable as phosphorus is one of the major nutrients contributing to the eutrophication of waterways and lakes, and is frequently the cause of water quality problems. The heretofore known methodologies for phosphorus removal from wastewater rely frequently on the chemical precipitation of phosphorus using hydrated aluminum sulfate, also known as alum. The present disclosure provides a methodology for removal phosphorus from a liquid waste product which does not involve chemical precipitation or the use of alum. Accordingly, the present disclosure further provides a method of disposing a liquid waste product substantially free of phosphorus obtainable from condensed distillers syrup, the method comprising: (a) providing a growth medium including a controlled substrate; (b) providing a microbial inoculum; (c) inoculating the growth medium with the microbial inoculum; (d) growing microbial cells under conditions that permit the conversion of the controlled substrate and producing bacterial mass and depleted growth medium; (e) separating the microbial mass from the depleted growth medium; (f) separating phosphorus from the depleted growth medium to obtain a phosphorus containing product and a liquid waste product substantially free of phosphorus; and (g) disposing the liquid waste product

In some embodiments, the liquid waste product comprises less than about 200 ppm phosphorus, less than about 150 ppm phosphorus, less than about 100 ppm phosphorus, less than about 50 ppm phosphorus, or less than about 10 ppm phosphorus.

In some embodiments, the liquid waste product, comprises less than about 200 ppm phosphorus, less than about 150 ppm phosphorus, less than about 100 ppm phosphorus, less than about 50 ppm phosphorus, or less than about 10 ppm phosphorus, and the liquid medium further comprises less than about 3 ppm dissolved oxygen, less than about 2 ppm, or less than about 1 ppm dissolved oxygen.

In some embodiments, the liquid waste product is disposed by discharge in a waterway. In other embodiments, the liquid waste product is disposed by underground injection.

In further embodiments of the present disclosure, there is provided a method for preparing a microbial inoculum comprising: (b) preparing a liquid medium comprising the controlled substrate; (c) inoculating the liquid medium with a microbial inoculum comprising a plurality of microbial species; (d) growing the microbial species in the liquid medium to form a microbial community enriched for microbial species capable of growth on the food, beverage or biofuel processor by-product or the controlled substrate; (e) isolating the microbial community; and (f) preparing a microbial inoculum for microbial production from the enriched microbial community.

In accordance with the foregoing, a liquid medium comprising a food, beverage or biofuel processor by product or a controlled substrate is inoculated with a plurality of microbial species. The plurality of microbial species is a microbial community such as can be found in the mixed liquor suspended solids at a wastewater treatment plant receiving food by-products such as those that derive from a brewery, wet corn miller, or a similar food , beverage or biofuel processing plant. Most preferably, the plurality of microbial species includes thermophiles, and species that can utilize cellulose, starch and mixtures thereof as nutrient sources.

Upon inoculation the microbial species are grown to form a microbial community. This is achieved by providing appropriate growing conditions, such as for example conditions which include providing dissolved oxygen greater than 1.0 mg/L, nitrogen (in the form of urea) and phosphorus (in the form of phosphoric acid) in a ratio of 100:1:2 (BOD:N:P) to the inoculated by-product or controlled substrate. Growth will generally be continued until such time that a more or less stable microbial community has formed. During the growth period the constitution of the microbial community changes. The relative quantity of bacterial species that is capable of efficiently using the controlled substrate or the food, beverage or biofuel processor by-product as a growth substrate is expected to increase, and the relative quantity of bacterial species that is unable to efficiently use the controlled substrate as a growth medium, is expected to decrease. A “stable a microbial community” in this regard is a microbial community wherein the relative quantities of microbial species present in the community remain approximately constant under the selected growing conditions.

In order to assess relative quantities of microbial species, the microbial community may be characterized, for example via genomic analysis, using a representative sample of nucleic acid material or other methods that allow the identification of the genera and/or species of microorganisms, characteristics of microbial physiologies, and quantitation of microorganisms in a microbial community.

In accordance herewith a microbial inoculum is prepared. The microbial inoculum may subsequently be used to inoculate a liquid medium comprising a controlled substrate for microbial production

The present disclosure further provides microbial inoculum for microbial production using a growth medium comprising a controlled substrate. In particular embodiments the controlled substrate is prepared from palm oil effluent, condensed syrup, glycerin, or vinasse.

In some embodiments, microbial production is used to prepare biomass for use in the preparation of a food or feed product. Such food and feed product may be prepared by processing and extraction of fractions from the microbial biomass. Processing techniques may vary widely and will be generally known to those in the art. Thus processing techniques include, without limitation, dewatering (optionally in the presence of a coagulant), the use of physical, chemical or enzymatic cell lysis techniques, drying, enzymatic treatment and preservation of the microbial mass. In some embodiments, cell lysis techniques are used to generate a biomass comprising lysed cells. In some embodiments, enzymatic treatment involves the treatment of biomass with nucleases in order to obtain a biomass comprising elevated levels of nucleotides. Fractionation techniques may also widely vary and may include, for example, filtration or ultrafiltration. Preferably extracted microbial fractions for the formulation of food and feed products include, without limitation the following: a protein fraction, a lipid fraction, a vitamin fraction, and a nucleic acid fraction, all of which may be used as ingredients to formulate food products for human consumption , as well as feed products for animal consumptions, e.g. cattle feed, pig feed, poultry feed and fish feed.

In some embodiments, the controlled substrate and/or the growth medium has a BOD ranging from about 100,000 mg BOD/L to about 500,000 mg BOD/L, including all values and subranges in between. In some embodiments, the controlled substrate is selected from the group the group consisting of condensed distillers syrup, glycerin, palm oil mill effluent (POME), biodiesel, vinasse, and combinations thereof. In some embodiments, the controlled substrate is condensed distillers syrup. In some embodiments, the controlled substrate is glycerin. In some embodiments, the controlled substrate is POMIE. In some embodiments, the controlled substrate is vinasse.

In some embodiments, the method further includes treating the growth medium to generate a treated growth medium. In some embodiments, the treated growth medium has a biological oxygen demand (BOD) value from about 10,000 mg BOD/L to about 90,000 mg BOD/L. In some embodiments, the treated growth medium has a biological oxygen demand (BOD) value from about 10,000 mg BOD/L to about 40,000 mg BOD/L. In some embodiments, the treated growth medium has a biological oxygen demand (BOD) value from about 15,000 mg BOD/L to about 25,000 mg BOD/L.

In some embodiments, each microbial species is independently selected from the group of microbial domains consisting of Bacteria, Archaea and Eucarya.

In some embodiments, each bacterial species is independently selected from the microbial domain consisting of Archaea, and the microbial species is an extremophile.

In some embodiments, each bacterial species is independently selected from the microbial domain consisting of Archaea, and the microbial species is an extremophilic microbial species classified as a thermophilic, a halophilic, an acidophilic or an alkaliphilic microbial species.

In some embodiments, the thermophilic microbial species belongs to the domain of Archaea and is a microbial species exhibiting optimal growth between 45° C. and 122° C. In some embodiments, the thermophilic microbial species belongs to the domain of Archaea and is a microbial species exhibiting optimal growth between 40° C. and 100° C. In some embodiments, the thermophilic microbial species belongs to the domain of Archaea and is a microbial species exhibiting optimal growth between 45° C. and 55° C.

In some embodiments, the halophilic microbial species belongs to the domain of Archaea and is a microbial species exhibiting optimal growth at NaCl concentrations of at least 0.2M.

In some embodiments, the acidophilic microbial species belongs to the domain of Archaea and is a microbial species exhibiting optimal growth at a pH of 3 or less.

In some embodiments, the alkaliphilic microbial species belongs to the domain of Archaea and is a microbial species exhibiting optimal growth at pH of 9 or more.

In some embodiments, each microbial species is independently selected from the group of microbial families consisting of Sphingobacteriaceae, Comamonadaceae, Xanthomonadaceae, Microbacteriaceae, Flavobacteriaceae, Alcaligenaceae, Porphyromonadaceae, and Saprospiraceae. In some embodiments, the controlled substrate is condensed distillers syrup, and each microbial species is independently selected from the group of microbia families consisting of Sphingobacteriaceae, Comamonadaceae, Xanthomonadaceae, Microbacteriaceae, Flavobacteriaceae, Alcaligenaceae, Porphyromonadaceae, and Saprospiraceae.

In some embodiments, each microbial species is independently selected from the group of microbial genera consisting of Lewinella, Parapedobacter, Emticicia, Luteibacter, Thermomonas, Denitrobacter, Comamonas, Chryseobacterium, Microbacterium, Dysgonomonas, Acinetobacter, and Curvibacter.

In some embodiments, the controlled substrate is condensed distillers syrup, and each microbial species is independently selected from the group of microbial genera consisting of Lewinella, Parapedobacter, Emticicia, Luteibacter, Thermomonas, Denitrobacter, Comamonas, Chryseobacterium, Microbacterium, Dysgonomonas, Acinetobacter, and Curvibacter.

In some embodiments, each microbial species is independently selected from the group of microbial species consisting of Lewinella marina, Parapedobacter koreensis, Emticicial oligotroghica, Luteibacter anthropi, Curvibacter gracilis, Dysgonomonas wimpennyi, and Thermomonas koreensis.

In some embodiments, the controlled substrate is condensed distillers syrup, and each microbial species is independently selected from the group of microbial species consisting of Lewinella marina, Parapedobacter koreensis, Emticicial oligotroghica, Luteibacter anthropi, Curvibacter gracilis, Dysgonomonas wimpennyi, and Thermomonas koreensis.

In some embodiments, the method further includes incubating the inoculated medium in a bioreactor under conditions suitable for microbial growth to generate an incubation product. In some embodiments, the conditions suitable for microbial growth include sterile or substantially sterile conditions, including conditions ensuring that no microbial species, other than those present in the inoculum, are permitted to grow or substantially grow during incubation. In some embodiments, substantially sterile conditions are maintained during incubation until growth of the microbial species present in the inoculum in the medium in the bioreactor is established. Thus, for example, sterile or substantially sterile conditions may be maintained during incubation until a least of one the microbial species present in the inoculum has established at least about 10%, 20%, 30%, 40% or 50% of the log number of bacteria obtained during the exponential growth phase. In other embodiments, the sterile or substantially sterile conditions are maintained during incubation until the incubation product is formed. Methodologies to determine quantities of microbial species present in a bioreactor are well known to the art, and include for example sampling and determining the number of colony forming units (cfus). In other embodiments, substantially sterile conditions are maintained until growth of the microbial species present in the inoculum in the medium in the bioreactor is established, and thereafter incubation is continued under non-sterile conditions.

In some embodiments, the conditions suitable for microbial growth include conditions involving removing a portion of the microbial mass from the bioreactor, and controlling the mean cell residence time or MCRT. Removal may be achieved using a continuous or discontinuous process, and typically the bioreactor volume is kept more or less constant by replenishing the bioreactor with fresh medium. The MCRT can be calculated by dividing the total mass of microbial organisms in the medium by the mass of the microbial organisms removed per unit time. The total mass of microbial organisms in the process can be measured by various conventional methods, for example by removing samples of known volume from the aerobic basins and clarifiers, filtering the microorganisms out of the wastewater sample using a membrane filter with a nominal pore size of approximately one micron, drying the filter and captured cells, calculating the mass of microbial organisms in the samples, and extrapolating the mass of the microbial organisms in the samples to the mass of the microbial organisms in the total volume present in the process. Thus, by way of example only, if the total mass of microbial organisms is 100 pounds, and 20 pounds of microbial organism mass is removed per day, the MCRT is 5 days. In preferred embodiments, the conditions suitable for microbial growth include maintaining an MCRT is about 30 days or less. In other embodiments, the MCRT is maintained to be about 6 days, about 5 days, about 4 days, about 3 days or about 2 days. Alternatively stated, in preferred embodiments no more than 1/6, 1/5, 1/4, 1/3, 1/2 per day of the microbial organism mass is removed from the microbial proliferation process. The MCRT may be optimized using methods as desired. Thus, the MCRT may be optimized, for example, by establishing the desired BOD removed, which may be for example 90% or more or 95%, and determining a corresponding MCRT.

Where methods and/or schematics described above indicate certain events and/or flow patterns occurring in certain order, the ordering of certain events and/or flow patterns may be modified. Additionally, certain events may be performed concurrently in parallel processes when possible, as well as performed sequentially.

What is claimed is:

1. A method, comprising:
receiving a growth medium including a controlled substrate;
treating the growth medium to generate a treated growth medium, the treated growth medium having a biological oxygen demand (BOD) value from about 10,000 mg BOD/L to about 90,000 mg BOD/L;
inoculating the treated growth medium with a microbial composition to generate an inoculated medium, the microbial composition including two or more microbial species, each microbial species independently selected from the group of microbial families consisting of Sphingobacteriaceae, Comamonadacea, Xanthomonadaceae, Microbacteriaceae, Flavobacteriaceae, Alcaligenaceae, Porphyromonadaceae, and Saprospiraceae;
incubating the inoculated medium under conditions suitable for microbial growth to generate an incubation product;
and extracting biomass from the incubation product.

2. The method of claim 1, wherein the controlled substrate is selected from the group of controlled substrates consisting of condensed distillers syrup, palm oil mill effluent, vinasse or glycerin.

3. The method of claim 1, wherein the treating of the growth medium including one or more of diluting the growth medium; or adding urea nitrogen to the growth medium; the adding urea nitrogen including adding urea nitrogen in an amount to achieve a BOD: nitrogen ratio of about 100:3 to 100:5 in the treated growth medium; the incubating the inoculated medium under conditions suitable for microbial growth including maintaining the pH of the inoculated medium between about 6.5 and about 7.5;

4. The method of claim 1, wherein each microbial species independently selected from the group of microbial genera consisting of Lewinella, Parapedobacter, Emticicia, Luteibacter, Thermomonas, Denitrobacter, Comamonas, Chiyseobacterium, Microbacterium, Dysgonomonas, Acinetobacter and Curvibacter; each microbial species independently selected from the group of microbial species consisting of Lewinella marina, Parapedobacter koreensis, Emticicial oligotroghica, Luteibacter anthropi, Curvibacter gracilis, Dysgonomonas wimpennyi, and Thermomonas koreensi.

5. The method of claim 1, wherein the extracting including separating a residual component from the incubation product to generate the biomass; further comprising treating subsequently received growth medium with the residual component, or a filtered portion thereof; further comprising sterilizing the biomass to generate a food product.

6. The method of claim 1, wherein the treated growth medium having a biological oxygen demand (BOD) ranging from about 10,000 mg BOD/L to about 40,000 mg BOD/L; further, the treated growth medium having a biological oxygen demand (BOD) ranging from about 15,000 mg BOD/L to about 25,000 mg BOD/L.

7. A method, comprising
receiving a growth medium including a controlled substrate;
treating the growth medium to generate a treated growth medium, the treated growth medium having a biological oxygen demand (BOD) ranging from about 10,000 mg BOD/L to about 1,000,000 mg BOD/L;
inoculating the treated growth medium with a microbial composition to generate an inoculated medium;
incubating the inoculated medium under conditions suitable for microbial growth to generate an incubation product, including maintaining the pH of the inoculated medium between about 6.5 and about 7.5;
and extracting biomass from the incubation product.

8. The method according to claim 7, wherein the conditions suitable for microbial growth further include maintaining the dissolved oxygen level at a concentration greater than approximately 3.0 mg /L.

9. The method according to claim 7, wherein the conditions suitable for microbial growth further include maintaining an MCRT of 3 days or less.

10. The method of claim 7, wherein the controlled substrate is selected from the group consisting of condensed distillers syrup, glycerin, palm oil mill effluent (POME), and vinasse.

11. The method according to claim 7, wherein the treating the growth medium including one or more of diluting the growth medium; or adding urea nitrogen to the growth medium; wherein the adding urea nitrogen including adding urea nitrogen in an amount to achieve a BOD:nitrogen ratio of about 100:3 to 100:5 in the treated growth medium.

12. The method of claim 7, wherein the conditions suitable for microbial growth during incubation are substantially sterile until microbial growth within the medium is established; wherein the microbial composition comprises a microbial species independently selected from the group of microbial domains consisting of Bacteria and Archaea; the microbial composition comprises a microbial species independently selected from the Archaea microbial domain and wherein the microbial species is an extremophile; the extremophile is selected from the group consisting of thermophilic, halophilic, acidophilic or alkaliphilic species.

13. The method of claim 7, wherein the controlled substrate is condensed distillers syrup, each microbial species independently selected from the group of microbial families consisting of Sphingobacteriaceae, Comamonadaceae, Xanthomonadaceae, Microbacteriaceae, Flavobacteriaceae, Alcaligenaceae, Porphyromonadaceae and Saprospiraceae; wherein, each microbial species independently selected from the group of microbial genera consisting of Lewinella, Parapedobacter, Emticicia, Luteibacter, Thermomonas, Denitrobacter, Comamonas, Chryseobacterium Microbacterium, Dysgonomonas, Acinetobacter and Curvibacter;
wherein, each microbial species independently selected from the group of microbial species consisting of Lewinella marina, Parapedobacter koreensis, Emticicia oligotrophica, Luteibacter anthropi, Curvibacter gracilis, Dysgonomonas wimpennyi, and Thermomonas koreensis.

14. The method of claim 7, wherein the extracting including separating a residual component from the incubation product to generate the biomass.

15. The method of claim 14, wherein it further comprising sterilizing the biomass to generate a food product; or/and lysing the biomass to generate a food product comprising microbial components comprising lysed cells; or/and contacting the biomass with nucleases to generate a food product comprising elevated levels of nucleotides; or/and treating subsequently received growth medium with the residual component, or a filtered portion thereof.

16. The method of claim 7, wherein the treated growth medium having a biological oxygen demand (BOD) ranging from about 10,000 mg BOD/L to about 40,000 mg BOD/L, further, the treated growth medium having a biological oxygen demand (BOD) ranging from about 15,000 mg BOD/L to about 25,000 mg BOD/L.

17. A method for producing phosphorus comprising: (a) providing a growth medium including a controlled substrate; (b) providing a microbial inoculum; (c) inoculating the growth medium with the microbial inoculum; (d) growing microbial cells under conditions that permit the conversion of the controlled substrate and producing bacterial mass and depleted growth medium; (e) separating the microbial mass from the depleted growth medium; and (f) extracting phosphorus from the depleted growth medium.

18. A method of disposing a liquid waste product substantially free of phosphorus obtainable from condensed distillers syrup, the method comprising: (a) providing a growth medium including a controlled substrate; (b) providing a microbial inoculum; (c) inoculating the growth medium with the microbial inoculum; (d) growing microbial cells under conditions that permit the conversion of the controlled substrate and producing bacterial mass and depleted growth medium; (e) separating the microbial mass from the depleted growth medium; (f) separating phosphorus from the depleted growth medium to obtain a phosphorus containing product and a liquid waste product substantially free of phosphorus; and (g) disposing the liquid waste product.

19. The method according to claim 18, wherein the controlled substrate is condensed distillers syrup.

20. The method according to claim 18, wherein the liquid waste product comprises less than 10 ppm phosphorus.
